(19) United States
(12) Patent Application Publication
(10) Pub. No.: US 2006/0216350 A1
(43) Pub. Date: Sep. 28, 2006

(54) Ganglionic blocking agents for the treatment of epithelial diseases

(76) Inventors: Gordon J. Dow, Santa Rosa, CA (US); Daniel A. Bucks, Millbrae, CA (US); Nayan Desai, Santa Rosa, CA (US)

Correspondence Address: HOWARD EISENBERG, ESQ., 2206 APPLEWOOD COURT, PERKASIE, PA, 18944, US

(21) Appl. No.: 11/089,713

(22) Filed: Mar. 24, 2005

(51) Int. Cl. A61K 31/44; A61K 9/14; A61K 31/14

(52) U.S. Cl. 424/484; 514/358; 514/643

(57) ABSTRACT

Compositions, and methods of making and using a composition, containing a ganglionic blocking agent and water provide increased penetration of the ganglionic blocking agent into and through epithelial surfaces, such as skin and mucosal surfaces, provide increased release from formulations containing such ganglionic blocking agents, and provide increased antiviral effectiveness compared with compositions containing a ganglionic blocking agents and lacking water.

### FIELD OF THE INVENTION

The present invention pertains to the field of topical therapies for the treatment of epithelial diseases, such as diseases of the skin and of mucosal surfaces such as the oral or vaginal mucosa. In particular the invention pertains to the field of topical application of a ganglionic blocking agent for the treatment of such diseases, such as virally caused diseases such as fever blisters, cutaneous warts, and genital warts.

### BACKGROUND OF THE INVENTION

Warts are skin disorders caused, in humans, by human papilloma virus (HPV), of which at least 100 different types have been identified. Warts are benign epithelial growths characterized by a greatly thickened and frequently hyperkeratinized epidermis. The warts typically present as discrete, skin colored, raised, and roughened lesions on the surface of the skin. Warts may also be flat, such as is typically the case when the warts are located on the soles of the feet, where they are referred to as plantar warts. Estimates of the prevalence of warts vary between 0.78% and 22% of the population. Thus, in the United States, between about 2.5 million to 66 million people suffer from warts.

Many warts, especially in children and adolescents, are self-limiting and will disappear without treatment over a period of months. In adults, however, warts typically do not disappear. Most warts are left untreated, unless the warts are unsightly or painful.

Treatment of warts is complicated by the thick, hyperplastic and hyperkeratinized tissue that presents a formidable barrier to the penetration of active pharmaceutical agents. Current treatment modalities include surgical, cryosurgical (freezing), or laser removal of the warts, application of chemical compounds such as cantharidin which cause the skin under a wart to blister, thus facilitating removal of the wart, and the application of caustic chemicals, such as salicylic acid, to the surface of the wart. The caustic chemicals exert their beneficial effect by a keratolytic activity that slowly destroys the virus-infected epidermis.

Salicylic acid is available as an over-the-counter medication for the treatment of warts. Application of salicylic acid causes a mild irritation and may stimulate an immune response that helps in removal of a wart. Salicylic acid has been shown to produce clearance of warts in 67% of patients with hand warts. Sterling, et al., British Journal of Dermatology, 144:4-11 (2001). No presently available therapy for warts has a very high success rate. Salicylic acid, although it is effective in clearing warts in only about two-thirds of patients, has been determined to be the most effective method for treating warts; even more effective than surgical techniques such as cryosurgery. Gibbs, et al., British Medical Journal (BMJ), 325:461-464 (2002).

A significant clinical need exists for a therapeutic medicament that can be applied as a therapy to the surface of a virally-induced lesion such as a wart, which therapy is effective, less irritating or preferably non-irritating, and which results in clearance of such lesions in a time that is less than the currently recommended duration of 12 week therapy with salicylic acid.

Baldone, U.S. Pat. Nos. 5,686,448; 4,902,720; and 5,158,980, discloses that ganglionic blocking agents (GBA), such as particular quaternary ammonium compounds, are effective in the treatment of viral infections. GBAs are drugs that block transmission in autonomic ganglia without producing any preceding or concomitant change in the membrane potentials of the ganglion cells. GBAs produce ganglionic blockade by occupying receptor sites on ganglion cells and stabilizing the postsynaptic membranes against the actions of acetylcholine liberated from presynaptic nerve endings. Baldone discloses that quaternary ammonium ganglionic blocking agents, such as tetraethylammonium chloride (TEAC), may be used to treat infections due to viruses from a variety of families, including papovaviruses.

Baldone discloses that the antiviral activity of TEAC is shared by all ganglionic blocking agents and that the antiviral activity of ganglionic blocking agents is effective against viruses as a whole and not just against herpes simplex virus (HSV), the virus that is exemplified in the Baldone patents. In the Examples, Baldone discloses effective systemic therapy in patients suffering from HSV lesions. Further, Baldone describes effective topical treatment of a cutaneous labialis HSV lesion when treated for five days with an ointment containing 1% TEAC in a hydrophilic ointment base.

Each of the Baldone patents cited herein is incorporated in this specification by reference.

Even though TEAC and the other ganglionic blocking agents have been shown to be relatively non-toxic and effective in the treatment of viral diseases, these agents have not gained acceptance for the treatment of warts or other cutaneous viral diseases. In a proof of principle clinical trial it was found that a 5% TEAC ointment in a hydrophilic ointment base is safe and effective in treating warts. However, even though the positive results were statistically significant, the overall clinical results showed a low level of clearing of warts.

A significant need exists for a formulation containing one or more ganglionic blocking agents that is more effective against epithelial diseases such as warts than are such presently known formulations.

### DESCRIPTION OF THE INVENTION

It has been surprisingly discovered that combining a ganglionic blocking agent in a composition with excipients known to increase penetration of therapeutic agents through skin or mucosal surfaces, such as propylene glycol, sodium lauryl sulfate, glycolic acid, oleic acid, oleyl alcohol, or urea, does not significantly increase the penetration of the ganglionic blocking agent through skin. Rather, it has been surprisingly discovered that combining a ganglionic blocking agent in a composition with water results in increased penetration of the ganglionic blocking agent through skin.

Thus, it has been discovered that combining water with a ganglionic blocking agent in a composition provides surprisingly better epithelial penetration of the ganglionic blocking agent, thus providing increased effectiveness in the treatment of conditions affecting epithelium or tissues situated beneath the epithelium. For example, the composition of the invention provides increased antiviral efficacy and increased effectiveness in the treatment of virally-induced skin and mucosal diseases. Additionally, the utility of the aqueous composition containing a ganglionic blocking agent can be further improved by the inclusion of a gelling agent in the composition. Additionally, the utility of such aqueous compositions can be improved by the inclusion of alcohol in the composition for certain applications of the invention. The aqueous compositions of the invention can be any of a variety of dosage forms, including but not limited to liquids, including solutions and suspensions, foams, and various semi-solids including lotions, creams, and gels, including hydrogels and hydro-alcoholic gels. The aqueous composition of the invention may be embodied as a patch for local or transdermal application, such as a hydrogel patch. Those skilled in the art will understand how to make and use the composition of the invention in such aqueous dosage forms.

In one embodiment, the invention is an aqueous pharmaceutical formulation for topical administration, which formulation contains water and a ganglionic blocking agent at a concentration effective to reduce the number and/or size of virally induced lesions, such as warts or fever blisters on the skin or mucosal surface of a subject suffering from such lesions. Preferably, the pharmaceutical composition contains a gelling or thickening agent. If desired, the pharmaceutical composition may contain an alcohol. The concentration of water in the composition is at least that which will provide an enhancement in epithelial penetration, such as skin or mucosal surface penetration, compared to a comparable composition that is substantially free of water. If desired, known penetration enhancing agents such as propylene glycol, sodium lauryl sulfate, glycolic acid, oleic acid, oleyl alcohol, or urea, may optionally be included in the composition.

In a preferred embodiment, the formulation is a fluid having a viscosity greater than that of water, such as a thickened liquid or a semi-solid. The liquid formulation of the invention may have a viscosity, for example, of that of water to about 10 centipoise (cP). The viscosity of such liquid formulation may be higher, if desired. A preferred viscosity is 1000 cP or higher, and a viscosity of up to 10,000 cP is more preferred for the thick liquid formulation of the invention. A semi-solid formulation of the invention typically has a viscosity of 10,000 cP to 300,000 cP or higher. Preferably, the semi-solid formulation of the invention has a viscosity between 20,000 cP and 200,000 cP and most preferably between 25,000 cP and 150,000 cP. Measurement of viscosity may be performed at 25° C. with any suitable viscometer or rheometer such as Brookfield rotational viscometers (Brookfield Engineering, Middleboro, Mass.), and using Viscosity Standard Fluids certified by methods traceable to the United States Institute of Standards and Technology, such as those available from Brookfield Engineering, as the reference viscosity to match.

In another embodiment, the invention is a method for making a pharmaceutical composition for topical administration, which methods includes combining water and a ganglionic blocking agent, wherein the concentration of the ganglionic blocking agent that is present in the composition is at a level that is effective to reduce the number or size of virally-induced lesions such as warts or fever blisters on the skin or mucosal surface of a subject suffering from such lesions, and wherein the concentration of water that is present in the composition is that which is sufficient to provide an enhancement in skin or mucosal penetration compared to a comparable composition that is substantially free of water. Preferably, a gelling agent is further combined with the water and the ganglionic blocking agent to form the preferred semi-solid embodiment of the invention as a gel. A cream or lotion embodiment of the invention can be made with or without such gelling agent. If desired, an alcohol is further combined with the water and the ganglionic blocking agent, and with the gelling agent if present. If desired, known penetration enhancing agents such as propylene glycol, sodium lauryl sulfate, glycolic acid, oleic acid, oleyl alcohol, or urea, may optionally be combined to make the composition.

In another embodiment, the invention is a method for treating a viral lesion, such as cutaneous or genital warts, genital herpes, or fever blisters. According to this embodiment, an effective amount of a pharmaceutical composition containing water and a ganglionic blocking agent is applied to the surface of a lesion on an individual suffering from such lesion in an amount and for a period of time effective to reduce the size and/or number of the lesions on the individual. The concentration of ganglionic blocking agent in the composition is that which is effective to reduce the number and/or size of such lesions on the skin or mucosal surface of an individual suffering therefrom. Preferably, the composition is a semi-solid aqueous composition. Preferably, the pharmaceutical composition contains a gelling agent. If desired, the pharmaceutical composition contains an alcohol. If desired, known penetration enhancing agents such as propylene glycol, sodium lauryl sulfate, glycolic acid, oleic acid, oleyl alcohol, or urea, may optionally be included in the composition. The concentration of water in the composition is at least that which will provide an enhancement in skin penetration compared to a comparable composition that is substantially free of water.

Any one or more ganglionic blocking agents that have anti-viral activity are suitable for the invention. Ganglionic blocking agents are well known in the art and the anti-viral activity of such agents may be determined by any method known in the art for testing anti-viral activity or by methods described in the Examples that follow.

Thus, the ganglionic blocking agent may be a quaternary ammonium compound, such as those that are disclosed in Baldone, U.S. Pat. No. 5,686,448. Not all quaternary ammonium compounds are ganglionic blocking agents. For example, benzalkonium chloride and dimethyl distearyl ammonium chloride are well known topical antiseptic agents or skin and hair conditioners used on various epithelial surfaces. The present invention pertains to ganglionic blocking agents and includes the small subset of quaternary ammonium compounds that have ganglionic blocking activity.

The quaternary ammonium ganglionic blocking agent may be a mono-quaternary ammonium compound. Examples of these compounds include tetraethylammonium halogenides, such as tetraethylammonium chloride (“TEAC”), triethyl-(β-4-stilbene oxyethyl)-ammonium iodide, 2,6-Dimetyl-1,1-diethylpeperidinium bromide, 2(p-Butoxyphenyl)-2-(methylene-N-morpholine)-dioxolan-( 1,3)-methylbromide, N-Phenacyl-O-kl-mandelyl-tropinium chloride, and d-3,4(1′3′-Dibenzyl-2′-ketoimidazolidino)-1,2-trimethylene-thiophanium d-camphor sulphonate. The quaternary ammonium ganglionic blocking agent may be a bis-quaternary ammonium compound. Examples of these compounds include hexane-1,6-bis-(trimethylammonium halogenide), such as hexane-1,6-bis-(trimethylammonium bromide) (“hexamethonium bromide”), hexane-1,6-bis-(ethyldimethylammonium bromide), pentane-1,5-bis-(trimethylammonium halogenide), N,N,N1,N,3-Pentamethyl-N′N′-diethyl-3-azapentane-1,5-diammonium halogenide, bis-β-diethylaminoethyl ethyl dimethyliodide, bis-(β-dimethylaminoethyl)-thioether diethyliodide, and pentamethylene-1,5-bis-(N-methylpyrrolidinium tartrate). The quaternary ammonium ganglionic blocking agent may be asymmetrical. Examples of these compounds include phenylethane-p-\acute{ω}-bis-(trimethylammonium iodide), N,N-Diethyl-N′,N′-dimethyl-bis-aminoethyl ether bis-ethyltartrate, 1-[β-(β′-Diethylaminoethoxy)-ethyl]-pyrrolidine bis-methiodide, diethylaminoethyl ester of 1,6-dimethylpipecholic acid bis-methiodide, diethylaminoethyl ester of 1-azabicyclo-[2,2,3]-octane-2-carboxylic acid bis methiodide, 4,5,6,7-tetrachlor-2-methyl-2{bis-trimethylammoniumethyl)-isoindolinium dichloride, N-(γ-Trimethlammonium propyl)-N-methylcamphidinium bis-methylsulphate, and N,N,N′-Trimethyl-N-(5-cyan-5-5-diphenylpentyl)-ethylene-1-ammonium-2-morpholinium dichloride or methylsulphate.

The ganglionic blocking agent of the invention may be a chemical compound other than a quaternary ammonium compound. Examples of such suitable compounds that are not quaternary ammonium compounds include tertiary amines, such as 2,3,3-Trimethyl-2-dimethylaminobutane hydrobromide, 1,2,2,6,6-Pentamethylpeperidinium tartrate, N,N,1,2,2-Pentamethylcyclohexylamine hydrobromide or hydrochloride, 3-Dimethylaminoisocamphane hydrobromide or hydrochloride, Tri-(β-diethylaminoethyl)-amine trihydrochloride, and d-sparteine hydroiodide, and secondary amines such as 3-methylaminoisocamphane hydrochloride and 2,6-dimethylpiperidine hydrochloride. Other ganglionic blocking agents include hexamethonium, tetraethylammonium, mecamylamine, emepronium, pentolinium, trimethidinium, chlorisondamine, trimethaphan, and pempidine.

A preferred ganglionic blocking agent for the invention is the quaternary ammonium compound hexamethonium, such as in the form of a halogenide, preferably hexamethonium bromide, referred to hereafter as HMB, or hexamethonium chloride, referred to hereafter as HMC. A more preferred ganglionic blocking agent for the invention is a tetraethylammonium halogenide, preferably tetraethylammonium chloride, referred to hereafter as TEAC. The invention is described using these two ganglionic blocking agents as representative illustrations, and particularly with respect to TEAC. It is understood, however, that such description is not restricted to these two agents but is applicable to any ganglionic blocking agent having anti-viral activity. A combination of two more ganglionic blocking agents or a combination of one or more ganglionic blocking agents with a drug substance from a different pharmacologic class represents another embodiment of the invention.

The concentration of ganglionic blocking agent in the composition of the invention is that which is effective to cause a decrease in the number and/or size of skin lesions, such as virally-induced lesions like warts, in a subject suffering from such lesions. The effective concentration may very depending on several parameters, including the indication for which the ganglionic blocking agent is used and the identity of the ganglionic blocking agent that is in the composition. For TEAC, the concentration of ganglionic blocking agent is typically about 1% w/w or higher and is preferably 5% w/w or higher More preferred is a concentration of 7% or higher such as at least about 10%. Even more preferred is a concentration between 10% and 40%, with a concentration of about 10% to 20% being most preferred. Concentrations higher than 40% may be used in the composition, if desired. All concentrations recited herein are percent by weight (% w/w), unless indicated otherwise.

The composition of the invention is aqueous, preferably a semi-solid, such as a gel, cream, or lotion. The cream or lotion may be of the oil-in-water or water-in-oil emulsion type. Such creams or lotions preferably contain a thickening agent which may be, for example, a gelling agent or a waxy thickening agent such as cetyl alcohol, stearyl alcohol, white wax, or glyceryl monostearate.

The concentration of water in the composition of the invention is that which is effective to provide an increase in skin penetration of the ganglionic blocking agent. Thus, the concentration of water in the composition of the invention is conceived to be between 1.5% and 99.5% w/w. Preferably, the concentration of water in the composition is about 5% to 95%, most preferably 10% w/w or higher. Thus, the concentration may be, for example, 1.5%, 5%, 10%, 15%, 20%, 25%, 30%, 35%, 40%, 45%, 50%, 55%, 60%, 65%, 70%, 75%, 80%, 85%, 90%, 95%, 99.5%, or any concentration between 1.5% and 99.5%. The concentration of water may be varied depending upon the identity and concentration of the ganglionic blocking agent and the presence and concentration of additional excipients in the composition as will be understood by those skilled in the art.

In a preferred embodiment, the composition of the invention is a semi-solid and the most preferred embodiment is a gel, which contains a gelling agent, which is preferably a polymeric gelling agent. Any gelling agent that is water-dispersible, is suitable for use on epithelial tissue, is compatible with the particular ganglionic blocking agent in the composition, and forms an aqueous gel of substantially uniform consistency, is suitable for use in the composition of the invention. One preferred gelling agent is hydroxypropylcellulose, such as that sold under the tradename KLUCEL® (Hercules Incorporated, Wilmington, Del., USA). Another preferred gelling agent is hydroxyethylcellulose, such as that sold under the tradename NATROSOL® (Hercules Incorporated). Other suitable gelling agents include carboxyvinyl polymers, such as CARBOPOL® 934, 940, and 941 (B.F. Goodrich Co., Akron, Ohio, USA), ETD 2020™, and ULTREZ® (Noveon, Inc., Cleveland, Ohio, USA). Additional suitable gelling agents are polyvinyl alcohol, polyethylene oxides, propylene glycol alginates, methylcellulose, hydroxypropylmethylcellulose and natural polymeric gums such as xanthan, and carrageenan. The concentration of gelling agent in the composition may be varied depending on several factors, including the desired viscosity of the gel composition. A wide variety of viscosities are contemplated concerning the semi-solids of the invention, ranging from a water-like consistency to a paste-like consistency. In a preferred embodiment, the aqueous gel of the invention contains hydroxypropylcellulose at a concentration between about 0.2% to 10% w/w.

The optional inclusion of alcohol in the gel compositions of the invention provides for a decreased drying time, reduced tackiness, and a potentially improved cosmetic elegance. Preferably, the alcohol is an alkyl alcohol. Preferred alkyl alcohols include ethyl alcohol (also known as EtOH, ethanol, and alcohol USP) and isopropyl alcohol. Because of their potential to be irritating, alkyl alcohols typically are not preferred for treatment of skin conditions with cracks and fissures or of conditions with mucous membrane involvement.

The concentration of alcohol in the gel compositions of the invention may be varied, depending for example on the identity of the alcohol included in the composition. For compositions of the invention other than gels, such as creams and lotions, the concentration of alcohol is typically 10% or less. Preferably, the concentration of alcohol is at a level at or above that which provides desirable skin-feel properties. In a gel containing alcohol, the concentration of alcohol in the composition is preferably about 5% w/w or more and more preferably about 10% w/w or more. If desired, the concentration of alcohol may be as high as 35%, 50%, or even 75% or more.

If desired, the composition of the invention may contain excipients in addition to those discussed above. Such excipients may include, for example, fragrances, dyes and pigments, preservatives, humectants, stabilizers such as anti-oxidants, keratolytic agents, emollients, thickeners, chelating agents, and additional solvents such as propylene glycol or polyethylene glycol 400. If present, the concentration of such excipients is preferably at a level that does not unduly reduce skin penetration of the ganglionic blocking agent.

The compositions of the invention may be made by methods that are well known to the formulation art to combine ingredients to obtain a pharmaceutical composition. Thus, the compositions of the invention may be made by combining, such as by mixing, water and one or more ganglionic blocking agents and any excipients, plus or minus alcohol. For example, a gel composition of the invention may be made by obtaining water, heating or cooling the water to an appropriate temperature depending on the nature of the gelling agent to be used, adding a gelling agent to the water, adding a ganglionic blocking agent such as TEAC before or after adding the gelling agent, and adding other excipients as desired, preferably added and mixed one at a time. The gelling agent and the ganglionic blocking agent should be completely mixed to homogeneity in the mixture. Complete mixing of all ingredients is preferred. Alcohol may be added, either before or after the gelling agent is added, in order to make a hydro-alcoholic gel.

The compositions of the invention may be used for the topical treatment of skin and mucosal lesions, such as lesions caused by viruses. It is conceived that the compositions of the invention are useful for treating lesions and disorders caused by a variety of viruses.

Thus, the compositions of the invention may be used for treating lesions and disorders caused by (1) dsDNA viruses, such as adenoviridae, herpesviridae such as simplexvirus and varicellovirus, papovaviridae such as polyomavirus and papillomavirus, and poxviridae; (2) ssDNA viruses, such as circoviridae, and parvoviridae; (3) dsRNA viruses, such as birnaviridae and reoviridae; (4) ssRNA viruses, such as astroviridae, caliciviridae, coronaviridae, flaviviridae, picornaviridae, and togaviridae; (5) (−) sense RNA viruses, such as filoviridae, paramyxoviridae, pneumoviridae, rhabdoviridae, arenaviridae, bunyaviridae, and orthomyxoviridae; (6) RNA reverse transcribing viruses such as retroviridae; (7) and DNA reverse transcribing viruses such as hepadnaviridae.

One indication for the composition of the invention is cutaneous warts. Other indications include genital warts, genital herpes, and fever blisters. Another indication for the composition of the invention is to administer a ganglionic blocking agent to cervical mucosa to treat HPV (human papillomavirus) infections that are the cause of cervical dysplasia, a common pre-cancerous condition in women. For such indications where the lesion to be treated is on or near a mucosal surface, because of the potential for irritation to mucosal surfaces, it is preferred that the composition have a high water content and contain little or no alcohol. For these areas, a semisolid preparation such as a viscous gel or cream is preferred. In the case of a gel, the formulation is preferably alcohol free and, in the case of a cream, the formulation preferably has a high water content, such as higher than 40%. For vaginal application, the pH of such formulations may be beneficially adjusted to a pH at or near the physiological vaginal acidity.

In accordance with the method of treatment of the invention, the aqueous composition of the invention containing a ganglionic blocking agent is topically applied to the surface of a wart or other lesion on the surface of the skin or mucous membrane of a subject in need thereof. The amount and frequency of application of the composition are that which are effective in reducing the size and/or number of the lesions on a subject suffering from such lesions and may vary depending on the severity of the condition in a particular subject and the identity and concentration of the ganglionic blocking agent in the composition.

The subject may be any mammal that is suffering from a skin or mucosal disorder that is amenable to treatment with a ganglionic blocking agent, such as a viral disease like warts. Thus, the subject may be a human or a lower mammal, such as a domesticated animal like a dog, a cat, or a horse.

In another embodiment of the invention, a kit is provided for the topical treatment of a virally-induced epithelial disorder, such as a skin or mucosal disorder. The kit contains a jar, tube, squeeze bottle, or other container containing a composition containing water and a ganglionic blocking agent. The composition is preferably a gel, such as an alcoholic gel, as described herein. Preferably, the kit further contains instructions for applying the composition topically to affected areas of the skin or mucosal surface to ameliorate the symptoms of the disorder. Preferably, the ganglionic blocking agent is TEAC and preferably, the aqueous composition is a gel, such as a hydro-alcoholic gel. The jar, tube, squeeze bottle, or other container is preferably packaged within a box upon which additional information, such as instructions, may be written.

The invention is further described in the following non-limiting examples. In the examples, the invention is illustrated in regards to the treatment of warts. It will be understood, however, that the invention is applicable to the treatment of other skin or mucosal disorders that are caused or exacerbated by viruses.

### EXAMPLE 1

### Preparation of Test Formulations

The following 6 formulations containing 5% TEAC were made by combining the ingredients indicated in Table 1.

TABLE 1

| | Formula 1 | Formula 2 | Formula 3 | Formula 4 | Formula 5 | Formula 6 |
|---|---|---|---|---|---|---|
| FORMULA | hydrophilic | hydrophilic | hydro- | cream | hydro- | hydro- |
| TYPE | ointment | ointment | alcoholic | | alcoholic | alcoholic |
| | | | gel | | gel | gel |
| AQUEOUS? | NO | NO | YES | YES | YES | YES |
| COMPOSITION | | | | | | |
| (% w/w) | | | | | | |
| TEAC | 5 | 5 | 5 | 5 | 5 | 5 |
| Aquaphor | q.s.ad 100 | | | | | |
| Glycerin | 3.15 | | | | | |
| Propylene | | 3.15 | 10 | 5 | 10 | 10 |
| Glycol | | | | | | |
| Urea | | 10 | | 5 | 10 | |
| Hydrophilic | | q.s.ad 100 | | | | |
| ointment | | | | | | |
| Hydroxypropyl | | | 2 | | 2 | |
| cellulose | | | | | | |
| (Klucel) | | | | | | |
| Ethyl alcohol | | | 50 | | 50 | 20 |
| Sodium lauryl | | | | 0.25 | 0.25 | |
| sulfate | | | | | | |
| Glycolic acid | | | 5 | | | |
| Emulsifying | | | | 10 | | |
| wax | | | | | | |
| Isopropyl | | | | 2 | | |
| myristate | | | | | | |
| Cetyl alcohol | | | | 1 | | |
| Stearyl alcohol | | | | 1 | | |
| Polyvinyl | | | | | | 12 |
| alcohol | | | | | | |
| Purified water | | | q.s.ad 100 | q.s.ad 100 | q.s.ad 100 | q.s.ad 100 |

Formulas 1 to 6 are compositions containing 5% TEAC. Formula 1 is a prior art occlusive anhydrous hydrophilic ointment (Aquaphor based). Formula 2 is an occlusive anhydrous hydrophilic ointment containing propylene glycol and urea. Formulas 3 to 6 are aqueous compositions of the invention. Formulas 3, 5, and 6 are hydro-alcoholic gel compositions and Formula 4 is a cream composition.

### EXAMPLE 2

### Efficacy of Prior Art Composition

The 5% TEAC composition of Formula 1 of Example 1 was tested in a double-blind placebo-controlled study for efficacy in the treatment of warts. Forty five (45) patients with warts were evaluated in the study. Thirty patients were in the active group treated with Formula 1, and 15 patients were in the vehicle group treated with the same ointment vehicle but without TEAC.

Warts present at day 1 were termed baseline warts. These warts were the basis of the primary efficacy determinations. Wart clearance was defined as resolution of the wart with complete healing of the treatment site. Complete response was defined as resolution of all baseline warts. Partial response was resolution of some but not all of baseline warts. No response was defined as all baseline warts still present.

There were 212 warts in the active group and 90 warts in the vehicle group for a total of 302 warts at baseline. Starting at Week 4 and continuing through the study, efficacy (% warts cleared) was statistically greater (p<0.02, Fisher's Exact Test) in the active group treated with the Formula 1 compared to the vehicle group treated with placebo. At week 12, after the last dose, 29% of warts cleared in the active group (151 of 212 warts remaining) compared to 9% of warts cleared (82 of 90 warts remaining) in the vehicle group.

Twelve of the 30 patients comprising the active group and 5 of the 15 patients in the vehicle group responded to treatment with some reduction in the total number of warts at week 12 compared to baseline. Thirteen percent (13%) of patients in the active group (4) compared to 7% of the patients in the vehicle group (1) experienced complete clearing of all warts at week 12. No patients in this study experienced an increase in the number of warts from their baseline level. Results of this study are summarized in Table 2.

TABLE 2

| | Treated with | | Treated | |
|---|---|---|---|---|
| | Formula 1 | | with Vehicle | |
| | (active group) | | (vehicle group) | |
| | | 12 week | | 12 week |
| | Baseline | treatment | Baseline | treatment |
| Total # of warts | 212 | 151 | 90 | 82 |
| % of baseline | 100 | 71 | 100 | 91 |
| % of patients cleared | 0 | 13 | 0 | 7 |
| % of warts cleared | 0 | 29 | 0 | 9 |

As shown in Table 2 and as described above, the ointment containing 5% TEAC was effective in treating warts compared to vehicle without TEAC. Twenty-nine (29) per cent of warts treated with the 5% TEAC ointment were cleared and 13% of patients with warts were cleared of warts. This result was found to be statistically significant (p<0.02) compared to the results obtained with vehicle alone, in which only 9% of warts were cleared and 7% of patients were cleared of warts.

Although the prior art 5% TEAC ointment was shown statistically to be effective in treating warts, the failure of the ointment to clear 71% of the warts in the active group indicates that the prior art ointment has a clinically low level of clearance of warts.

### EXAMPLE 3

### Skin Penetration Studies

All 6 TEAC-containing compositions (Formulas 1 to 6) of Example 1 were made with tracer levels (˜1 μCi/dose) of ¹⁴C-labeled TEAC. A single clinically relevant dose (4 mg formulation/cm²) was applied to dermatomed human skin obtained from elective surgery. Each study used abdominal skin from a single human donor. Five samples were tested for each of the formulas. Percutaneous absorption was evaluated using the skin mounted on diffusion cells to evaluate the amount of TEAC delivered to the epidermis. The receptor fluid consisted of phosphate buffered saline containing 0.1% sodium azide and 1.5% Oleth 20. Following a 24-hour exposure, residual formulation residing on the skin surface was removed by wiping with two dry cotton swabs. The upper layers of the stratum corneum were removed from the epidermis with a single cellophane tape-strip. The remaining epidermis was then physically separated from the dermis. Quantity of radioactivity in the tape-strip, epidermis, and total epidermis (single tape-strip plus epidermis) samples was determined using liquid scintillation counting techniques.

Data is presented in Table 3. The data is an average of the five skin samples on separate diffusion cells for each formula. The values of Table 3 are in percent of applied dose.

TABLE 3

| | Single | | Total |
|---|---|---|---|
| | Tape-strip | Epidermis | Epidermis |
| FORMULATION | (%) | (%) | (%) |
| Formula 1 | 1.14 | 3.23 | 4.37 |
| Formula 2 | 1.57 | 4.68 | 6.25 |
| Formula 3 | 8.07 | 20.75 | 28.38 |
| Formula 4 | 3.95 | 13.32 | 17.27 |
| Formula 5 | 2.78 | 4.94 | 7.73 |
| Formula 6 | 6.59 | 21.75 | 28.34 |

The data in Table 3 shows that in vitro human skin penetration of TEAC into the epidermis from each of the aqueous compositions of Formulas 3, 4, and 6 was 4 to 6.5 times as high as that from the prior art composition of Formula 1. Skin penetration from Formula 5, which contains a high level of urea which was shown to reduce penetration (see Example 4), was 1.75 times as high as that from the composition of Formula 1. Skin penetration was 2.75 to 4.5 times as high from the compositions of Formulas 3, 4, and 6 as from Formula 2, which is a hydrophilic ointment containing urea and propylene glycol. Formula 5, which is an aqueous composition, showed epidermal penetration about 1.25 times greater than Formula 2, even though both of these formulations contain a high concentration of urea. The data in Table 3 further shows that epidermal levels of TEAC from each of the aqueous compositions of Formulas 3 to 6 were higher than that for either of the anhydrous compositions of Formula 1 or 2.

### EXAMPLE 4

### Effect of Additional Excipients

A study was conducted as described in Example 3 to determine the potential enhancing effect on skin penetration of putative penetration enhancers propylene glycol, sodium lauryl sulfate, glycolic acid, and urea which are included in the compositions of Formulas 3 to 6 of Example 1. In order to determine the effect on skin penetration of these excipients, alone and in combination, nine different TEAC hydro-alcoholic gel formulations, denoted Formulations A to J, were made containing varying levels of these excipients according to a statistical design, and each formulation was tested for skin penetration. The formulations tested are shown in Table 4. All values in Table 4 are percent by weight (% w/w).

TABLE 4
TEAC formulations containing varying concentrations of excipients

| | FORMULATION | | | | | | | | | |
|---|---|---|---|---|---|---|---|---|---|---|
| | A | B | C | D | E | F | G | H | I | J |
| TEAC | 5 | 5 | 5 | 5 | 5 | 5 | 5 | 5 | 5 | 15 |
| Ethyl alcohol | 30 | 30 | 30 | 30 | 30 | 30 | 30 | 30 | 50 | 47.5 |
| Hydroxypropyl | 2.5 | 2.5 | 2.5 | 2.5 | 2.5 | 2.5 | 2.5 | 2.5 | 2 | 2.5 |
| cellulose | | | | | | | | | | |
| Propylene glycol | 2 | 20 | 2 | 20 | 2 | 20 | 2 | 20 | 10 | 10 |
| Sodium lauryl | 0 | 0 | 0 | 0 | 2.5 | 2.5 | 2.5 | 2.5 | 0.25 | 2.5 |
| sulfate | | | | | | | | | | |
| Glycolic acid | 0 | 18 | 18 | 0 | 18 | 0 | 0 | 18 | 0 | 15 |
| Urea | 0 | 0 | 20 | 20 | 0 | 0 | 20 | 20 | 10 | 0 |
| Purified water | 60.5 | 24.5 | 22.5 | 22.5 | 40 | 40 | 38 | 2 | 22.75 | 7.5 |

The data from the study established the following concerning a hydro-alcoholic gel composition of the invention. Urea decreases epidermal, dermal, or receptor fluid (skin penetration) levels (p<0.05, unpaired t-test). Sodium lauryl sulfate may increase epidermal drug level at high (20%) propylene glycol concentrations (p<0.05). Increasing the propylene glycol concentrations decreases epidermal, dermal, and receptor fluid (skin penetration) levels (p<0.05). Glycolic acid decreases dermal and receptor fluid (skin penetration) levels (p<0.05).

From this study, it is concluded that addition of urea or glycolic acid did not enhance skin deposition or penetration of TEAC from gel compositions. In addition, increasing propylene glycol concentration and addition of sodium lauryl sulfate did not significantly improve TEAC delivery over the formulation containing a low concentration of propylene glycol, no sodium lauryl sulfate, and no glycolic acid.

### EXAMPLE 5

### Effect on Penetration of Increasing Levels of Alcohol and of Ganglionic Blocking Agent

The effects of variation in ethyl alcohol and TEAC concentrations in prototype hydro-alcoholic gel formulations of the invention were evaluated on the in vitro percutaneous absorption of ¹⁴C-labeled TEAC following a 24-hour exposure using excised human skin as described in Example 3. The results are summarized in Table 5.

As shown in Table 5, varying ethanol concentration produced comparable TEAC deposition and penetration.

TABLE 5
In vitro percutaneous absorption of ¹⁴C-TEAC

| | Single | | Total |
|---|---|---|---|
| | Tape Strip | Epidermis | Epidermis |
| FORMULATION | (%) | (%) | (%) |
| 15% TEAC | 13.69 | 41.49 | 55.18 |
| 35% EtOH | | | |
| 15% TEAC | 13.16 | 39.21 | 52.37 |
| 50% EtOH | | | |
| 15% TEAC | 11.04 | 44.43 | 55.47 |
| 75% EtOH | | | |

Values are in percent of applied dose
N=5 to 6 cells per formulation

### EXAMPLE 6

### Comparison of Total GBA Skin Penetration from Formulations of the Invention and from Formulations of the Prior Art

A percutaneous penetration study was conducted in which two formulations containing 5% active ingredient (TEAC) and one formulation containing 1% TEAC were compared. One of the 5% formulations was a prior art formulation containing TEAC in a hydrophilic ointment based on Aquaphor (as described by Baldone) which contained no added water. The formulation of the invention was a hydro-alcoholic gel containing hydroxypropyl cellulose 1.8%, propylene glycol 5%, sodium lauryl sulfate 0.25%, ethyl alcohol 50%, tetraethyl ammonium chloride 5%, and purified water 37.95% to 41.95%. The results summarized in Table 5A are the averages of 6 samples and the amounts are presented as the total drug mass absorbed per square centimeter of skin in 24 hours at 32° C. Skin penetration was determined as described in Example 3. Data is shown in Table 5A.

TABLE 5A

| | Single | | Dermis | | Total |
|---|---|---|---|---|---|
| | Tape-strip | Epidermis | (ug/ | Receptor | (ug/ |
| FORMULATION | (ug/cm²) | (ug/cm²) | cm²) | (ug/cm²) | cm²) |
| Hydrophilic | 5.83 | 4.92 | 0.75 | 1.44 | 7.11 |
| Ointment - 5% | | | | | |
| (prior art) | | | | | |
| Hydro-alcoholic | 25.58 | 69.59 | 30.31 | 98.63 | 198.53 |
| Gel - 5% | | | | | |
| (invention) | | | | | |
| Hydro-alcoholic | 3.78 | 13.61 | 6.33 | 10.92 | 30.86 |
| Gel - 1% | | | | | |
| (invention) | | | | | |

The data of Table 5A shows that the penetration of active ingredient (TEAC) from the hydro-alcoholic gel of the invention is 27 times more than that from the prior art ointment at the same concentration. Even at one fifth the concentration of active ingredient, the gel formulation of the invention delivers more than 4 times the amount of drug into skin as does the prior art formulation. Thus, in contrast to the prior art, the formulation of the invention is capable of providing total human skin transport (epidermis, dermis, and receptor) of 10 micrograms or more of active ingredient per square centimeter over a twenty-four hour period.

### EXAMPLE 7

### In Vitro Release Studies

Seven (7) formulations designated 6A to 6G, each containing about 1% of a ganglionic blocking agent (GBA), were made by combining the ingredients indicated in Table 6. The ingredients in Table 6 are percent by weight (% w/w). Formulations 6A to 6C contain TEAC. Formulations 6D and 6E contain hexamethonium chloride. Formulations 6F and 6G contain pempidine, a non-quaternary ammonium GBA. Formulations 6C, 6E, and 6G are not aqueous compositions according to the invention.

TABLE 6

| | FORMULATION | | | | | | |
|---|---|---|---|---|---|---|---|
| | 6A | 6B | 6C | 6D | 6E | 6F | 6G |
| TEAC | 1.11 | 1.11 | 1.11 | | | | |
| Hexamethonium | | | | 1.00 | 1.00 | | |
| chloride | | | | | | | |
| Pempidine | | | | | | 1.00 | 1.00 |
| Purified water | 93.99 | 58.99 | | 94.10 | | 62.20 | |
| Aquaphor | | | 95.79 | | 95.90 | | |
| Ethanol | | 35.00 | | | | 35.00 | |
| Hydroxyethylcellulose | 1.80 | | | 1.80 | | | |
| Hydroxy- | | 1.80 | | | | 1.80 | |
| propylcellulose | | | | | | | |
| Glycerin | 3.10 | 3.10 | 3.10 | 3.10 | 3.10 | | 3.10 |

The formulations shown in Table 6 were studied to demonstrate in vitro release of GBA from water-containing formulations of the invention compared to formulations not containing water. Franz static diffusion cells (15 mm diameter orifice, O-ring joint, Crown Bio Scientific, Clinton, N.J., USA) were mounted on 9-cell manifolds and maintained at a constant temperature of 32° C. by use of recirculating water baths. These cells have an opening with a nominal area of 1.767 cm² and a receptor compartment with a volume ranging from 12 to 13 ml. Each diffusion cell was assembled by placing a support membrane (TUFFRYN® Membrane Filter, HT-450, 25 mm diameter, 0.45 μm pore size, Gelman Sciences Inc., Ann Arbor, Mich., USA) and then a TEFLON® (E.I. duPont de Nemours and Co., Wilmington, Del., USA) O-ring, which rested in a groove of the receptor side, the bottom half, of the diffusion cell. The donor side, top half, of the diffusion cell was then placed on top of the support membrane and held in place by use of a pinch clamp. The joint between the donor and receptor sides of each cell was wrapped with PARAFILM® (American Can Co., Greenwich, Conn., USA) to prevent evaporation of the receptor solution within the cell. Each cell was then filled with receptor solution containing degassed 50% ethanol in deionized water, taking care to dispel any air bubbles from under the membrane. The receptor fluid was continuously stirred using a TEFLON® magnetic stir bar and an inoculating loop cut to 6.0 cm from the top of the loop.

The membrane was allowed to impregnate with the receptor fluid for 1 hour prior to application of one of the test formulations 6A to 6G. An infinite dose (about 1.5 ml) of each formulation was applied onto the membrane using a syringe. Each formulation was applied in an alternating fashion to 4 diffusion cells. The diffusion cell donor compartment and the sampling port were sealed with PARAFILM® to prevent evaporation. At 2, 4, and 6 hours after application, the entire receptor solution was collected through the sampling port using a syringe fitted with TEFLON® tubing on the needle. The receptor compartment was then refilled with fresh receptor solution maintained at 32° C. The collected receptor fluid samples were then analyzed for the presence and concentration of TEAC, hexamethonium chloride, or pempidine, as appropriate, via ion-pairing chromatography.

The average cumulative amount of TEAC, hexamethonium chloride, and pempidine released into the receptor fluid from the four samples of each formulation is shown in Table 7. Of the seven samples, the highest rate of release of GBA was observed with hexamethonium chloride in water-based gel, formulation 6D, at 3595 μg/hr^{1/2}. The TEAC in water-based gel, formulation 6A, was only slightly lower. The release rate of TEAC from a water-based gel, formulation 6A, was 3472 μg/hr^{1/2}, and of TEAC from an alcoholic gel, formulation 6B, was 2921 μg/hr^{1/2}. Both TEAC and hexamethonium chloride release from non-aqueous ointment formulations 6C and 6E were below the limit of detection. The rate of release of pempidine from an alcoholic gel and a non-aqueous ointment, formulations 6F and 6G, were 2532 μg/hr^{1/2} and 304 μg/hr^{1/2}, respectively.

TABLE 7

| | Mean Release Rate | |
|---|---|---|
| | μg/hr^{1/2} | RANK |
| Formulation 6A - TEAC in | 3472 | 2 |
| water-based gel | | |
| Formulation 6B - TEAC in | 2921 | 3 |
| hydro-alcoholic gel | | |
| Formulation 6C - TEAC in | undetectable | 7 |
| non-aqueous ointment | | |
| Formulation 6D - HMC in | 3595 | 1 |
| water-based gel | | |
| Formulation 6E - HMC in | undetectable | 7 |
| non-aqueous ointment | | |
| Formulation 6F - pempidine | 2532 | 4 |
| in hydro-alcoholic gel | | |
| Formulation 6G - pempidine | 204 | 5 |
| in non-aqueous ointment | | |

The results of this study demonstrate that the release rate of GBA from water-containing formulations was significantly greater (p<0.05) than from anhydrous ointment formulations. The highest release rate was obtained with water-based non-alcoholic gels followed by water-based alcoholic gels. The release rate from each of the water-based gels for each GBA tested was at least 15 times higher than the highest release rate observed from any of the tested anhydrous formulations. These surprising advantageous results were obtained with ganglionic blocking agents that are quaternary ammonium compounds, such as TEAC and hexamethonium chloride, as well as with pempidine, which is a non-charged non-quaternary ammonium ganglionic blocking agent.

### EXAMPLE 8

### In Vivo Antiviral Efficacy

A study was performed to demonstrate the increased antiviral activity of an aqueous composition of the invention containing a ganglionic blocking agent compared to a prior art non-aqueous composition. The compositions were also compared to placebo treatment and to non-treatment. Additionally, a dose response curve for the aqueous compositions of the invention was generated by comparing antiviral activity for varying concentrations of the aqueous compositions of the invention. The ingredients in each of the formulations that were studied are listed in Table 8.

TABLE 8

| | Non- | | 1% | 5% | 15% | 30% | 15% | 15% | 15% |
|---|---|---|---|---|---|---|---|---|---|
| | aqueous | | TEAC | TEAC | TEAC | TEAC | TEAC | TEAC | HMC |
| | 5% | | Gel | Gel | Gel | Gel | Gel | Gel | Gel |
| Ingredients | TEAC | | 50% | 50% | 50% | 50% | 0% | 10% | 50% |
| % w/w | Ointment | Placebo | EtOH | EtOH | EtOH | EtOH | EtOH | EtOH | EtOH |
| TEAC | 5.48 | | 1.09 | 5.48 | 16.44 | 32.88 | 16.44 | 16.44 | |
| monohydrate | | | | | | | | | |
| Aquaphor | 91.37 | | | | | | | | |
| Glycerin | 3.15 | | | | | | | | |
| Ethanol USP | | 50 | 50 | 50 | 50 | 50 | | 10 | 50 |
| 200 proof | | | | | | | | | |
| Propylene | | 5 | 5 | 5 | 5 | 5 | 5 | 5 | 5 |
| Glycol | | | | | | | | | |
| Hydroxpropyl | | 1.8 | 1.8 | 1.8 | 1.8 | 1.8 | 1.8 | | 1.8 |
| cellulose | | | | | | | | | |
| Hydroxyethyl | | | | | | | | 1.8 | |
| cellulose | | | | | | | | | |
| Sodium | | 0.25 | 0.25 | 0.25 | 0.25 | 0.25 | 0.25 | 0.25 | 0.25 |
| lauryl sulfate | | | | | | | | | |
| Purified water | | 42.95 | 41.86 | 37.47 | 26.51 | 10.07 | 76.51 | 66.51 | 27.51 |
| Hexamethonium | | | | | | | | | 17.24 |
| chloride | | | | | | | | | |

Thirty New Zealand White rabbits of both genders were purchased from Covance Research Products, Inc. (Denver, Pa., USA). The rabbits weighed between 2.0 and 2.5 kg when purchased and grew to between 4 and 5 kg by the end of the study. During the treatment portion of the study, the rabbits weighed between 2.5 and 3.5 kg.

The rabbits were divided into 6 groups of 5 rabbits. Four (4) treatment sites of 1 cm² on the back of each rabbit, with two sites on the right side of the back and 2 sites on the left side of the back, were inoculated with a dosage of Cottontail rabbit papillomavirus (CRPV) stock known to be sufficient to induce the formation of papillomas. The CRPV model, first described in Shope and Hurst, J. Exp. Med., 58:607-624 (1933), has been widely used to test to test the antiviral efficacy of chemical compounds and has been accepted as an animal model for human papillomavirus (HPV) related disease.

Treatment began on day 5 following CRPV infection and consisted of once-daily topical application of 100 μl of a test formulation to each site for 5 days per week for 8 weeks. The treatment groups were dosed as shown in Table 9. Each rabbit received identical treatment on each of the two sites on the same side of its back.

TABLE 9

| Group | Left Side Treatment | Right Side Treatment |
|---|---|---|
| A | Topical Placebo | 30% TEAC gel |
| | (50% EtOH) | (50% EtOH) |
| B | 15% TEAC gel | 1% TEAC gel |
| | (50% EtOH) | (50% EtOH) |
| C | 15% TEAC gel | No Treatment |
| | (10% EtOH) | |
| D | 15% TEAC gel | 5% TEAC ointment |
| | (0% EtOH) | |
| E | 15% Hexamethonium | No Treatment |
| | Chloride (50% EtOH) | |
| F | 5% TEAC gel | No Treatment |
| | (50% EtOH) | |

The ability of each of the GBA test formulations to inhibit the development of papillomas following infection was visually assessed for 7 weeks beginning on day 12 following papillomavirus inoculation. Antiviral activity associated with each formulation of GBA and treatment group is summarized in Table 10. The results indicate the presence or absence of papillomas at each site.

TABLE 10

| | Papillomas/Sites Treated after Inoculation | | | | |
|---|---|---|---|---|---|
| Formulation | Day 33 | Day 40 | Day 47 | Day 54 | Day 61 |
| 15% TEAC gel | 1/10 | 3/10 | 2/10 | 4/10 | 1/10 |
| (0% EtOH) | | | | | |
| 15% TEAC gel | 0/10 | 0/10 | 0/10 | 1/10 | 0/10 |
| (50% EtOH) | | | | | |
| 1% TEAC gel | 8/10 | 10/10 | 10/10 | 9/10 | 9/10 |
| (50% EtOH) | | | | | |
| 5% TEAC gel | 0/10 | 0/10 | 1/10 | 1/10 | 1/10 |
| (50% EtOH) | | | | | |
| 30% TEAC gel | 0/10 | 0/10 | 0/10 | 1/10 | 0/10 |
| (50% EtOH) | | | | | |
| 15% Hexamethonium | 1/10 | 3/10 | 3/10 | 4/10 | 4/10 |
| chloride (50% EtOH) | | | | | |
| 15% TEAC gel | 3/10 | 2/10 | 3/10 | 3/10 | 0/10 |
| (10% EtOH) | | | | | |
| Placebo gel | 10/10 | 10/10 | 10/10 | 9/10 | 9/10 |
| 5% TEAC ointment | 10/10 | 10/10 | 10/10 | 10/10 | 10/10 |
| No Treatment | 25/30 | 27/30 | 27/30 | 27/30 | 28/30 |

The data in Table 10 shows the following. The placebo gel did not express significant antiviral activity relative to the untreated inoculation sites. The 5% TEAC ointment (non-aqueous) expressed no antiviral activity. Papilloma growth following application of the ointment was comparable to placebo gel and untreated inoculated sites. In contrast, the 5% TEAC aqueous gel expressed significant, almost complete, antiviral activity and was significantly better (p<0.05) than the placebo gel and the 5% TEAC ointment at every observation point. Even at 1%, the TEAC aqueous gel expressed antiviral activity. The 15% and 30% TEAC aqueous gels significant antiviral activity unrelated to the concentration of alcohol in the gels. The 15% hexamethonium aqueous gel expressed significant antiviral activity relative to placebo gel and the TEAC ointment.

The results establish that aqueous gel formulations containing a ganglionic blocking agent have significantly better antiviral activity in vivo than do similar non-aqueous formulations and that, for TEAC, a classic dose response curve exists with maximum effectiveness obtained with a 15% TEAC concentration.

The contralateral site dosing regimen employed in the study precludes antiviral activity observed to be due to systemic absorption of the GBA. In Group A, the two treatment sites on the left side of the rabbits were dosed with placebo gel and the right side treatment sites were dosed with the highest concentration of TEAC gel, 30%. Group A rabbits received the highest body burden of TEAC, 21.6 mg TEAC/kg/day. However, the placebo gel treated sites in group A had comparable levels of papilloma growth to the untreated sites in Groups E and F. Group E rabbits were not dosed with TEAC formulations and Group F received the lowest body burden of TEAC, 3.6 mg TEAC/kg/day. Yet, the treatment sites contralateral to the Placebo gel and untreated sites dosed with TEAC gels expressed dramatic and statistically significant antiviral activity. It is especially noteworthy that the treatment sites contralateral to the 5% TEAC ointment dosed with 15% TEAC gel expressed dramatic and statistically significant antiviral activity whereas the 5% TEAC ointment did not express antiviral activity in this animal model.

### EXAMPLE 9

The composition of the invention includes preferred semi-solid dosage forms such as creams and lotions. Creams and lotions of the invention may be compositions having a water phase dispersed in an oil phase. Alternatively, they may be oil phase dispersed in an aqueous phase. The dispersion of one phase in the other is aided by any of the emulsifiers well known in the art of emulsions. The oil phase may be a blend of any of numerous water-immiscible components such as mineral oil, isopropyl myristate, diisopropyl adipate, silicones, emulsifying wax, cetyl alcohol, stearyl alcohol. The emulsions may be prepared by separately mixing the aqueous phase components together and the oil phase components together and then emulsifying the two mixtures by mixing at elevated temperatures.

Non-limiting examples of a lotion and a cream in accordance with the invention are shown in Tables 11 and 12.

### Example of a Preferred Lotion Formulation

TABLE 11

| | Percent by weight |
|---|---|
| Ingredient | (% w/w) |
| Ganglionic Blocking Agent | 1-30 |
| Mineral Oil | 2-10 |
| C12-14 Alkyl Benzoate | 2-5 |
| Cetyl Alcohol | 0.5-2.0 |
| Steareth-2 | 0.5-2.0 |
| Steareth-21 | 0.5-3.0 |
| Hydroxypropylmethyl-Cellulose | 0-0.5 |
| Glycerin or Propylene Glycol | 2-10 |
| Methylparaben | 0.17 |
| Propylparaben | 0.03 |
| Citric Acid or Sodium Hydroxide | q.s. pH |
| Purified Water | q.s.ad 100 |

### Example of a Preferred Cream Formulation

TABLE 12

| | Percent by weight |
|---|---|
| Ingredient | (% w/w) |
| Ganglionic Blocking Agent | 1-30 |
| Cyclomethicone | 1-5 |
| Isopropyl Myristate | 2-10 |
| Stearyl Alcohol | 1-5 |
| Xanthan Gum | 0.0-0.8 |
| Magnesium Aluminum Silicate | 0-0.5 |
| PEG-8 Stearate | 2-5 |
| PEG-40 Stearate | 1-3 |
| Preservatives | 0.2 |
| Citric Acid or Sodium Hydroxide | q.s. pH |
| Purified Water | q.s.ad 100 |

### EXAMPLE 10

A non-limiting example of a solution formulation in accordance with the invention is shown in Table 13.

### Example of a Preferred Solution Formulation

TABLE 13

| | Percent by weight |
|---|---|
| Ingredient | (% w/w) |
| Ganglionic Blocking Agent | 1-50 |
| Glycerin or Propylene Glycol | 3-10 |
| Ethyl Alcohol | 0-50 |
| Preservatives | 0.2 |
| Citric Acid or Sodium Hydroxide | q.s. pH |
| Purified Water | q.s.ad 100 |

If desired, a low level of a thickening agent, such as 0.1 to 0.3% hydroxyethyl cellulose, hydroxypropyl methylcellulose, or polyvinyl alcohol may be added to the formulation of Table 13.

### EXAMPLE 11

A non-limiting example of a gel formulation in accordance with the invention is shown in Table 14.

### Example of a Preferred Gel Formulation

TABLE 14

| | Percent by weight |
|---|---|
| Ingredient | (% w/w) |
| Ganglionic Blocking Agent | 1-50 |
| Glycerin or Propylene Glycol | 3-10 |
| Gelling Agent | 0.2-10 |
| Ethyl Alcohol | 0-50 |
| Preservatives | 0.2 |
| Citric Acid or Sodium Hydroxide | q.s. pH |
| Purified Water | q.s.ad 100 |

The prior art has shown that ganglionic blocking agents, such as TEAC, are active in humans to treat viral diseases and lesions, such as warts due to human papilloma virus and diseases due to herpes simplex virus. As shown herein, the ability of GBAs to penetrate skin is enhanced by combining a GBA in a composition containing water. As shown herein, the release rate of a GBA from a pharmaceutical composition is increased when the composition contains water. Additionally, as shown herein, the antiviral efficacy of a GBA in an aqueous composition is dramatically increased compared to that of a GBA in a non-aqueous composition. These significant improvements over the prior art are not dependent on the presence in a composition of ingredients other than water, such as alcohol. The present invention represents a major advance in the development of therapeutic formulations for the topical treatment of lesions or diseases due to viral infection.

Further modifications, uses, and applications of the invention described herein will be apparent to those skilled in the art. It is intended that such modifications be encompassed in the following claims.

1. A pharmaceutical formulation comprising a ganglionic blocking agent and water.

2. The pharmaceutical formulation of claim 1 wherein the concentration of water in the formulation is sufficient to provide an enhancement in skin or mucosal penetration compared to a comparable composition that is free of water.

3. The pharmaceutical formulation of claim 1 which provides total human skin transport of 10 micrograms or more of active ingredient per square centimeter over a twenty-four hour period.

4. The pharmaceutical formulation of claim 1 wherein the concentration of water in the formulation is 5% w/w or higher.

5. The pharmaceutical formulation of claim 1 which is a semi-solid.

6. The pharmaceutical formulation of claim 1 which comprises a gelling agent.

7. The pharmaceutical formulation of claim 6 which further comprises an alcohol.

8. The pharmaceutical formulation of claim 1 wherein the ganglionic blocking agent is a quaternary ammonium compound.

9. The pharmaceutical formulation of claim 8 wherein the quaternary ammonium compound is selected from the group consisting of mono-quaternary ammonium compound, bis-quaternary ammonium compound, and asymmetrical quaternary ammonium compound.

10. The pharmaceutical formulation of claim 9 wherein the quaternary ammonium compound is a hexamethonium or tetraethylammonium compound.

11. The pharmaceutical formulation of claim 1 wherein the ganglionic blocking agent is a chemical compound other than a quaternary ammonium compound.

12. The pharmaceutical formulation of claim 11 wherein the ganglionic blocking agent is selected from the group consisting of a tertiary amine, a secondary amine, hexamethonium, tetraethylammonium, mecamylamine, emepronium, pentolinium, trimethidinium, chlorisondamine, trimethaphan, and pempidine.

13. The pharmaceutical formulation of claim 1 wherein the ganglionic blocking agent is tetraethylammonium chloride at a concentration of 5% w/w or higher.

14. A method for making a pharmaceutical formulation comprising combining a ganglionic blocking agent and water.

15. The method of claim 14 wherein the concentration of water in the formulation is sufficient to provide an enhancement in skin or mucosal penetration compared to a comparable composition that is free of water.

16. The method of claim 14 wherein the formulation provides total human skin transport of 10 micrograms or more of active ingredient per square centimeter over a twenty-four hour period.

17. The method of claim 14 which further comprises combining a gelling agent with the ganglionic blocking agent and water.

18. The method of claim 17 which further comprises combining alcohol with the ganglionic blocking agent and water.

19. A method for treating a disorder in a mammalian subject comprising topically applying to an epithelial surface a composition comprising a ganglionic blocking agent and water.

20. The method of claim 19 wherein the concentration of water in the composition is sufficient to provide an enhancement in skin or mucosal penetration compared to a comparable composition that is free of water.

21. The method of claim 19 wherein the formulation provides total human skin transport of 10 micrograms or more of active ingredient per square centimeter over a twenty-four hour period.

22. The method of claim 19 wherein the disorder is a viral disease or lesion.

23. The method of claim 22 wherein the viral disease or lesion is due to infection with a papilloma virus.

24. The method of claim 23 wherein the viral disease or lesion is cutaneous warts.

25. The method of claim 19 wherein the composition further contains a gelling agent.

26. The method of claim 25 wherein the composition further contains an alcohol.

27. The method of claim 19 wherein the concentration of water in the composition is 5% w/w or higher.

28. The method of claim 19 wherein the ganglionic blocking agent is a quaternary ammonium compound.

29. The method of claim 28 wherein the quaternary ammonium compound is a tetraethylammonium or hexamethonium compound.

30. A kit for topically treating a virally-induced epithelial disorder comprising a container containing a composition comprising water and a ganglionic blocking agent.

31. The kit of claim 30 wherein the concentration of water in the composition is sufficient to provide an enhancement in skin or mucosal penetration compared to a comparable composition that is free of water.

32. The kit of claim 30 wherein the composition provides total human skin transport of 10 micrograms or more of active ingredient per square centimeter over a twenty-four hour period.

33. The kit of claim 30 wherein the composition further contains a gelling agent.

34. The kit of claim 33 wherein the composition further contains an alcohol.

35. The kit of claim 30 wherein the ganglionic blocking agent is a quaternary ammonium compound.

36. The kit of claim 35 wherein the quaternary ammonium compound is a tetraethylammonium or hexamethonium compound.
